(19) United States
(12) Patent Application Publication
(10) Pub. No.: US 2009/0098046 A1
(43) Pub. Date: Apr. 16, 2009

(54) Combination Cancer Therapy with Anti-PSMA Antibodies

(76) Inventors: Michael Becker, Princeton, NJ (US); William Goeckeler, Princeton, NJ (US)

Correspondence Address: MORGAN LEWIS & BOCKIUS LLP, 1111 PENNSYLVANIA AVENUE NW, WASHINGTON, DC, 20004, US

(21) Appl. No.: 11/795,268

(22) PCT Filed: Jan. 17, 2006
(86) PCT No.: PCT/US06/01143

§ 371 Date: Apr. 24, 2008

Related U.S. Application Data
(60) Provisional application No. 60/643,589, filed on Jan. 14, 2005.

(51) Int. Cl. A61K 51/10; A61K 39/395; A61K 49/00; A61P 35/00; A61K 101/00; A61K 101/02; A61K 103/10; A61K 103/20; A61K 103/32; A61K 103/30

(52) U.S. Cl. 424/1.53; 424/139.1; 424/1.49; 424/9.1; 424/1.11

(57) ABSTRACT

This invention includes compositions and methods for combination cancer treatments, particularly involving at least one cytotoxic agent used in combination with an anti-PSMA monoclonal antibody.

### BACKGROUND OF THE INVENTION

Prostate cancer is among the most significant medical problems in the United States, as the disease is now the most common malignancy diagnosed in American males. The American Cancer Society estimates that for the year 2000, 180,400 new cases of prostate cancer were diagnosed with 31,900 deaths from the disease. Five year survival rates for patients with prostate cancer range from 88% for those with localized disease to 29% for those with metastatic disease. The rapid increase in the number of cases appears to result in part from an increase in disease awareness as well as the widespread use of clinical markers such as the secreted proteins prostate-specific antigen (PSA) and prostatic acid phosphatase (PAP) (Chiaroda (1991) Cancer Res. 51, 2498-2505).

The prostate gland is a site of significant pathology affected by conditions such as benign growth (BPH), neoplasia (prostatic cancer) and infection (prostatitis). Prostate cancer represents the second leading cause of death from cancer in man (Chiaroda (1991) Cancer Res. 51, 2498-2505). However the prostate is the leading site for cancer development in men. The difference between these two facts relates to prostatic cancer occurring with increasing frequency as men age, especially in the ages beyond sixty at a time when death from other factors often intervenes. Also, the spectrum of biologic aggressiveness of prostatic cancer is great, so that in some men following detection the tumor remains a latent histologic tumor and does not become clinically significant, whereas in other it progresses rapidly, metastasizes and kills the patient in a relatively short two to five year period (Chiaroda (1991) Cancer Res. 51, 2498-2505; Warner et al. (1991) Urologic Clinics of North America 18, 25-33).

In prostate cancer cells, two specific proteins that are made in very high concentrations are prostatic acid phosphatase (PAP) and prostate specific antigen (PSA) (Henttu et al. (1989) Bioch. Biophys. Res. Comm. 160, 903-908; Nguyen et al. (1990) Clin. Chem. 35, 1450-1455; Yong et al. (1991) Cancer Res. 51, 3748-3752). These proteins have been characterized and have been used to follow response to therapy. With the development of cancer, the normal architecture of the gland becomes altered, including loss of the normal duct structure for the removal of secretions and thus the secretions reach the serum. Measurement of serum PSA is suggested as a potential screening method for prostatic cancer. Indeed, the relative amount of PSA and/or PAP in the cancer changes as compared to normal or benign tissue.

PAP was one of the earliest serum markers for detecting metastatic spread (Nguyen et al. (1990) Clin. Chem. 35, 1450-1455). PAP hydrolyses tyrosine phosphate and has a broad substrate specificity. Tyrosine phosphorylation is often increased with oncogenic transformation. It has been hypothesized that during neoplastic transformation there is less phosphatase activity available to inactivate proteins that are activated by phosphorylation on tyrosine residues. In some instances, insertion of phosphatases that have tyrosine phosphatase activity has reversed the malignant phenotype.

PSA is a protease and it is not readily appreciated how loss of its activity correlates with cancer development (Henttu et al. (1989) Bioch. Biophys. Res. Comm. 160, 903-908; Yong et al. (1991) Cancer Res. 51, 3748-3752). The proteolytic activity of PSA is inhibited by zinc. Zinc concentrations are high in the normal prostate and reduced in prostatic cancer. Possibly the loss of zinc allows for increased proteolytic activity by PSA. As proteases are involved in metastasis and some proteases stimulate mitotic activity, the potentially increased activity of PSA could be hypothesized to play a role in the tumors metastases and spread (Liotta (1986) Cancer Res. 46, 1-7). Both PSA and PAP are found in prostatic secretions. Both appear to be dependent on the presence of androgens for their production and are substantially reduced following androgen deprivation.

Prostate-specific membrane antigen (PSMA) which appears to be localized to the prostatic membrane has also been identified as a marker for prostate cancer. This antigen was identified as the result of generating monoclonal antibodies to a prostatic cancer cell, LNCaP (Horoszewicz et al. (1993) Cancer Res., 53, 227-230). LNCaP is a cell line established from the lymph node of a hormone refractory, heavily pretreated patient (Horoszewicz et al. (1983) Cancer Res. 43, 1809-1818). This cell line was found to have an aneuploid human male karyotype. It maintained prostatic differentiation functionality in that it produced both PSA and PAP. It possessed an androgen receptor of high affinity and specificity. Mice were immunized with LNCaP cells and hybridomas were derived from sensitized animals. A monoclonal antibody was derived and was designated 7E11-C5 (Horoszewicz et al. (1993) Cancer Res. 53, 227-230). The antibody staining was consistent with a membrane location and isolated fractions of LNCaP cell membranes exhibited a strongly positive reaction with immunoblotting and ELISA techniques.

This monoclonal antibody was also used for detection of immunoreactive material in serum of prostatic cancer patients (Horoszewicz et al. (1993) Cancer Res., 53, 227-230). The immunoreactivity was detectable in nearly 60% of patients with stage D-2 disease and in a slightly lower percentage of patients with earlier stage disease, but the numbers of patients in the latter group were small. Patients with benign prostatic hyperplasia (BPH) were negative. Patients with no apparent disease were negative, but fifty to 60% of patients in remission yet with active stable disease or with progression demonstrated positive serum reactivity. Patients with non prostatic tumors did not show immunoreactivity with 7E11-C5.

The 7E11-C5 monoclonal antibody is now used as a molecular imaging agent and is the first and currently the only commercial product targeting PSMA. Prostascint® consists of 7E11-C5 linked to the radioisotope Indium-111. Due to the selective expression of PSMA by prostate cancer cells, Prostascint® can image the extent and spread of prostate cancer using a common gamma camera. U.S. Pat. No. 5,162,504 discloses and claims the monoclonal antibody 7E11-C5 and the hybridoma cell line that produces it. U.S. Pat. Nos. 4,671,958; 4,741,900 and 4,867,973 disclose and claim antibody conjugates, methods for preparing such conjugates, methods for using such conjugates for in vivo imaging, testing and therapeutic treatment, and methods for delivering radioisotopes by linking them to such antibodies.

### SUMMARY OF THE INVENTION

The invention encompasses a method for treating cancer which comprises a malignant cell expressing PSMA in a patient in need thereof comprising administering a monoclonal antibody or antigen binding fragment thereof which specifically binds to a cytoplasmic epitope on PSMA in combination with at least one cytotoxic agent.

In some embodiments the cytotoxic agent is administered prior to administration of the monoclonal antibody while in other embodiments it is administered simultaneously with the monoclonal antibody. In yet another embodiment the antibody is linked to a cytotoxic agent.

The invention also encompasses a method of imaging a tumor in a patient comprising administering a cytotoxic agent followed by administration of a monoclonal antibody which specifically binds to a cytoplasmic epitope on PSMA expressed by a malignant cell. In one embodiment the cytotoxic agent disrupts the malignant cell membrane and/or induces cellular apoptosis. In another embodiment, the monoclonal antibody binds to PSMA expressed by apoptotic endothelial cells.

In some embodiments of the invention, the anti-PSMA monoclonal antibody is 7E11-C5. In another embodiment of the invention, the patient is human.

In some embodiments of the invention the cytotoxic agent is selected from the group consisting of cytotoxins, chemotherapeutic agents and radiation. Examples of cytotoxins, include but are not limited to, gelonin, ricin, saponin, pseudomonas exotoxin, pokeweed antiviral protein, diphtheria toxin and complement proteins. Examples of chemotherapeutic agents, include but are not limited to, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens.

Additional examples of chemotherapeutic agents, include but are not limited to, BCNU, cisplatin, gemcitabine, hydroxyurea, paclitaxel, temozomide, topotecan, fluorouracil, vincristine, vinblastine, procarbazine, dacarbazine, altretamine, cisplatin, methotrexate, mercaptopurine, thioguanine, fludarabine phosphate, cladribine, pentostatin, fluorouracil, cytarabine, azacitidine, vinblastine, vincristine, etoposide, teniposide, irinotecan, docetaxel, doxorubicin, daunorubicin, dactinomycin, idarubicin, plicamycin, adriamycin, mitomycin, bleomycin, tamoxifen, flutamide, leuprolide, goserelin, aminoglutethimide, anastrozole, amsacrine, asparaginase, mitoxantrone, mitotane and amifostine.

In yet another embodiment where the cytotoxic agent is radiation, the radiation is a radioisotope. Examples of radioisotopes include, but are not limited to, ³H, ¹⁴C, ¹⁸F, ¹⁹F, ³¹P, ³²P, ³⁵S, ¹³¹I, ¹²⁵I, ¹²³I, ⁶⁴Cu, ¹⁸⁷Re, ¹¹¹In, ⁹⁰Y, ^{99m}Tc, ¹⁷⁷Lu. In one embodiment, the radioisotope is linked to the antibody by α-(5-isothiocyanato-2-methoxyphenyl)-1,4,7,10-tetraazacyclododecane-1,4,7,10-tetraacetic acid (methoxy-DOTA). Another example of radiation is external beam radiation.

Types of cancer that can be treated or imaged by the methods of the invention include solid tumors. Examples of solid tumors include, but are not limited to, endothelial cell carcinoma. Examples of endothelial cell carcinoma include, but are not limited to, renal cell carcinoma, colon carcinoma, transitional cell carcinoma, lung carcinoma, breast carcinoma and prostatic adenocarcinoma.

Examples of renal cell carcinoma include, but are not limited to, clear cell carcinoma, papillary carcinoma, chromophobe carcinoma, collecting duct carcinoma and unclassified carcinoma. Examples of lung carcinoma include, but are not limited to, adenocarcinoma, alveolar cell carcinoma, squamous cell carcinoma, large cell and small cell carcinoma. Examples of breast carcinoma include, but are not limited to, adenocarcinoma, ductal carcinoma in situ, lobular carcinoma in situ, invasive ductal carcinoma, medullary carcinoma and mucinous carcinoma.

Another example of solid tumor treatable by the methods of the invention includes endothelial cell sarcoma. In one embodiment, the sarcoma is a soft tissue sarcoma. Metastatic tumors are also treatable by the methods of the invention.

### DETAILED DESCRIPTION

This invention relates to combination cancer therapy, particularly involving at least one cytotoxic agent used in combination with a monoclonal antibody which binds to the cytoplasmic domain of Prostate Specific Membrane Antigen (PSMA). In one aspect, the invention includes compositions and methods for inducing apoptosis in a cancer cell or retarding the growth of a tumor by first administering a cytotoxic agent and subsequently administering an anti-PSMA monoclonal antibody. In this aspect of the invention, the cytotoxic agent disrupts the cancer cell(s), thereby expressing the cytoplasmic domain on the PSMA antigen. Exposure of the cytoplasmic domain allows for targeting of the remaining cancer cells with the anti-PSMA monoclonal antibody. In one aspect, the anti-PMSA monoclonal antibody is also linked to a cytotoxic agent capable of ablating the surrounding cancer cells not previously damaged by administration of the first cytotoxic agent.

In another aspect, the invention includes compositions and methods for inducing apoptosis in a cancer cell by administering to a cancer cell one or more cytotoxic agents in combination with an anti-PSMA monoclonal antibody. The present invention therefore includes a method of retarding the growth of a tumor by administering an anti-PSMA monoclonal antibody simultaneously with one or more cytotoxic agents. The simultaneous or subsequent administration of an anti-PSMA monoclonal antibody may also have the effect of reducing the amount of cytotoxic agent necessary for successful treatment thus reducing the severe side effects associated with cytotoxic agents such as chemotherapeutics and radiation.

### Combination Compositions

This invention includes pharmaceutical compositions for the treatment of abnormal cell growth in a mammal, including a human, comprising an amount of an anti-PSMA monoclonal antibody, in combination with a cytotoxic agent, that is effective in enhancing the effects of an the cytotoxic agent, and a pharmaceutically acceptable carrier. Generally, the cytoxic agent will damage the cancer cells in such a manner as to expose the cytoplasmic domain of PSMA. The antibody then binds to the exposed epitope (i.e., cytoplasmic domain) and can be used to target previously or subsequently administered cytotoxic agents (e.g., associated with the antibody) to the surrounding cancer cells which have not been damaged by the cytotoxic agent.

As used herein, the term “abnormal cell growth” unless otherwise indicated, refers to cell growth that is independent of normal regulatory mechanisms (e.g., loss of contact inhibition). This includes the abnormal growth and/or proliferation of cells in malignant or neoplastic diseases. Monoclonal antibody-dependent inhibition of abnormal cell growth can occur by a variety of mechanisms including, but not limited to, cell death, apoptosis, inhibition of cell division, transcription, translation, transduction, etc.

In one embodiment, the abnormal cell growth is cancer; particularly a cancer that involves malignant cells which express PSMA. As used herein, the term “cancer” unless otherwise indicated, refers to diseases that are characterized by uncontrolled, abnormal cell growth and/or proliferation. In one aspect of the invention, the cancer comprises a solid tumor including, but not limited to, metastatic solid tumors. In one aspect the solid tumor is an endothelial cell carcinoma, including, but not limited to, renal cell carcinoma, colon carcinoma, transitional cell carcinoma, lung carcinoma, breast carcinoma and prostatic carcinoma. Examples of renal cell carcinoma include, but are not limited to, clear cell carcinoma, papillary carcinoma, chromophobe carcinoma, collecting duct carcinoma and unclassified carcinoma. Examples of lung carcinoma include, but are not limited to, adenocarcinoma, alveolar cell carcinoma, squamous cell carcinoma, large cell and small cell carcinoma. Examples of breast carcinoma include, but are not limited to, adenocarcinoma, ductal carcinoma in situ, lobular carcinoma in situ, invasive ductal carcinoma, medullary carcinoma and mucinous carcinoma. In another aspect of the invention, the solid tumor is an endothelial cell sarcoma, including but not limited to, soft tissue sarcoma.

Examples of prostate carcinoma include, but are not limited to, prostatic adenocarcinoma small cell carcinoma, mucinous carcinoma, endometrioid cancer (prostatic ductal carcinoma), transitional cell cancer, squamous cell carcinoma, basal cell carcinoma, adenoid cystic carcinoma (basaloid), and signet-ring cell carcinoma.

As discussed above, the invention includes a pharmaceutical composition for the treatment of abnormal cell growth in a mammal, including a human, which comprises an amount of a monoclonal antibody which binds to a cytoplasmic domain of PSMA, as defined above, in combination with at least one chemotherapeutic agent and a pharmaceutically acceptable carrier.

The term “antibody” (Ab) as used herein includes monoclonal antibodies, polyclonal antibodies, multispecific antibodies (e.g., bispecific antibodies), single chain antibodies and antibody fragments, including antibody fragments or a CDR fused to a carrier protein, so long as they exhibit the desired biological activity, including but not limited to, epitope binding. In one embodiment, the desired biological activity is binding to an epitope on PSMA, including but not limited to, an epitope in the cytoplasmic domain of PSMA. The term “immunoglobulin” (Ig) is used interchangeably with “antibody” herein.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous components. In preferred embodiments, the antibody will be purified to greater than 95% by weight of antibody, and most preferably more than 99% by weight. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains (an IgM antibody consists of five of the basic heterotetramer unit along with an additional polypeptide called J chain, and therefore contain ten antigen binding sites, while secreted IgA antibodies can polymerize to form polyvalent assemblages comprising two to five of the basic four chain units along with J chain). The L chain from any vertebrate species can be assigned to one of two clearly distinct types, called kappa and lambda, based on the amino acid sequences of their constant domains and the methods of the current invention include the use of antibodies with either a kappa or lambda L chain. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. The gamma and alpha classes are further divided into subclasses on the basis of relatively minor differences in C_{H} sequence and function, e.g., humans express the following subclasses: IgG1, IgG2, IgG3, IgG4, IgA1 and IgA2. The methods of the present invention include the use of antibodies, including monoclonal antibodies, from any of the above classes and/or subclasses.

As used herein, the term “variable” refers to the fact that certain segments of the variable domains differ extensively in sequence among antibodies. The variable domain mediates antigen binding and define specificity of a particular antibody for its particular antigen. However, the variability is not evenly distributed across the 110-amino acid span of the variable domains. Instead, the variable regions consist of relatively invariant stretches called framework regions (FR) of about fifteen to thirty amino acids separated by shorter regions of extreme variability called “hypervariable regions” that are each about nine to twelve amino acids long. The variable domains of native heavy and light chains each comprise four framework regions, largely adopting a beta-sheet configuration, connected by three hypervariable regions, which form loops connecting, and in some cases forming part of, the beta-sheet structure. The hypervariable regions in each chain are held together in close proximity by the framework region and, with the hypervariable regions from the other chain, contribute to the formation of the antigen-binding site of antibodies (see Kabat et al. (1991) Sequences of Proteins of Immunological Interest, Public Health Service, National Institutes of Health). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” which contributes to the specificity of the antibody.

The term “monoclonal antibody” as used herein refers to an antibody obtained from a population of substantially homogeneous antibodies, i.e., the individual antibodies comprising the population are identical except for possible naturally occurring mutations that may be present in minor amounts and includes antibody fragments as defined herein. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al. (1975) Nature, 256, 495 or may be made using recombinant DNA methods in bacterial, eukaryotic animal or plant cells (see U.S. Pat. No. 4,816,567). The “monoclonal antibodies” may also be isolated from phage antibody libraries using the techniques described in Clackson et al. (1991) Nature, 352:624-628 and Marks et al. (1991) J. Mol. Biol. 222, 581-597, for example.

In one embodiment of the invention, the monoclonal antibody binds to an epitope on the cytoplasmic domain of a protein specific to cancer cells (i.e., a cancer cell marker). In another embodiment, the monoclonal antibody includes, but is not limited to, a monoclonal antibody which binds to an epitope on the cytoplasmic domain of PSMA, including but not limited to, the 7E11-C5 monoclonal antibody as described in U.S. Pat. No. 5,162,504 herein incorporated by reference in its entirety. The hybridoma cell line which produces the 7E11-C5 monoclonal antibody has been deposited with the American Type Culture Collection under Deposit No. HB10494.

The monoclonal antibodies used in the methods of the invention include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567 and Morrison et al. (1984) Proc. Natl. Acad. Sci. USA 81, 6851-6855). Chimeric antibodies of interest herein include, but are not limited to “humanized” antibodies comprising variable domain antigen-binding sequences derived from a non-human mammal (e.g., murine) and human constant region sequences.

As used herein, an “intact” antibody is one which comprises an antigen-binding site as well as a CL and at least heavy chain constant domains, C_{H1} and C_{H2} and C_{H3}. The constant domains may be native sequence constant domains (e.g., human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fv, Fab′ and F(ab′)₂ fragments; diabodies; linear antibodies (see U.S. Pat. No. 5,641,870 and Zapata et al. (1995) Protein Eng. 8, 1057-1062); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H1}). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H1} domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

As used herein, “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

As used herein, “Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding (see Rosenburg et al. (1994) The Pharmacology of Monoclonal Antibodies, Springer-Verlag, pp. 269-315).

As used herein, the term “diabodies” refers to small antibody fragments prepared by constructing sFv fragments (see preceding paragraph) with short linkers (about 5 to 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, WO 93/11161 and Hollinger et al. (1993) Proc. Natl. Acad. Sci. USA 90, 6444-6448.

As used herein, the term “cytotoxic agent” includes, but is not limited to agents which disrupt the membrane of a cancer cell to expose the cytoplasmic domain of PSMA. Examples include, but are not limited to, cytotoxins, chemotherapeutic agents and radiation, including radioisotopes and external beam radiation.

As used herein, the term “chemotherapeutic agent” unless otherwise indicated, refers to any agent used in the treatment of cancer which inhibits, disrupts, prevents or interferes with abnormal cell growth and/or proliferation. Examples of chemotherapeutic agents include, but are not limited to, agents which induce apoptosis, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, steroid hormones and anti-androgens. In some embodiments, the monoclonal antibody can be combined with a single species of chemotherapeutic agent while in other embodiments, it can be combined with multiple species of chemotherapeutic agents.

Examples of alkylating agents include, but are not limited to, carmustine, lomustine, cyclophosphamide, ifosfamide, mechlorethamine and streptozotocin. Examples of antibiotics include, but are not limited to, adriamycin, bleomycin, dactinomycin, daunorubicin, doxorubicin, idarubicin and plicamycin. Examples of anti-metabolites include, but are not limited to, cytarabine, fludarabine, 5-fluorouracil, 6-mercaptopurine, methotrexate and 6-thioguanine. Examples of mitotic inhibitors include, but are not limited to, navelbine, paclitaxel, vinblastine and vincristine. Examples of steroid hormones and anti-androgens include, but are not limited to, aminoglutethimides, estrogens, flutamide, goserelin, leuprolide, prednisone and tamoxifen.

Examples of pharmaceutical formulations of the above chemotherapeutic agents include, but are not limited to, BCNU (i.e., carmustine, 1,3-bis(2-chloroethyl)-1-nitrosurea, BiCNU®), cisplatin (cis-platinum, cis-diamminedichloroplatinum, Platinol®), doxorubicin (hydroxyl daunorubicin, Adriamycin®), gemcytabine (difluorodeoxycytidine, Gemzar®), hyrdoxyurea (hyroxycarbamide, Hydrea®), paclitaxel (Taxol®), temozolomide (TMZ, Temodar®), topotecan (Hycamtin®), fluorouracil (5-fluorouracil, 5-FU, Adrucil®), vincristine (VCR, Oncovin®) and vinblastine (Velbe® or Velban®).

In some aspects, the invention includes a population of conjugate molecules, said conjugate molecules comprising at least one monoclonal antibody which binds to a cytoplasmic domain of PSMA or a binding fragment thereof and at least one cytotoxic agent, wherein the extent of conjugation of monoclonal antibody and the agent is such that the effect of the agent in a mammal receiving the conjugate may be enhanced when compared to mixtures of the agent with monoclonal antibody, or the agent alone. In another aspect, the invention includes compositions comprising a population of conjugate molecules wherein at least one monoclonal antibody is conjugated to at least one cytotoxic agent and a pharmaceutically acceptable excipient. In some embodiments, the monoclonal antibody or a binding fragment thereof can be conjugated to a single species of cytotoxic agent while in other embodiments, it can be conjugated to multiple species of cytotoxic agents.

Pharmaceutical compositions of the present invention can be administered via parenteral, subcutaneous, intravenous, intramuscular, intraperitoneal, transdermal or buccal routes. For example, an agent may be administered locally to a tumor via microinfusion. Alternatively, or concurrently, administration may be by the oral route. For example, a chemotherapeutic agent could be administered locally to the site of a tumor, followed by oral administration of at least one monoclonal antibody which binds to the cytoplasmic domain of PSMA. The prior administration of the chemotherapeutic agent followed by the monoclonal antibody may have the effect of reducing the amount of chemotherapeutic agent necessary in subsequent treatments for successful outcomes, thus reducing the severe side effects associated with chemotherapeutic agents. The dosage administered will be dependent upon the age, health, and weight of the recipient, kind of concurrent treatment, if any, frequency of treatment, and the nature of the effect desired.

The present invention further includes compositions containing one or more monoclonal antibodies which bind to the cytoplasmic domain of PSMA or binding fragments thereof and one or more cytotoxic agents that are useful in the treatment of cancer. While individual needs vary, determination of optimal ranges of effective amounts of each component is within the skill of the art. Typical dosages comprise 1.0 pg/kg body weight to 100 mg/kg body weight. The preferred dosages for systemic administration comprise 100.0 ng/kg body weight to 10.0 mg/kg body weight. The preferred dosages for direct administration to a site via microinfusion comprise 1 ng/kg body weight to 1 mg/kg body weight.

In addition to the monoclonal antibodies and cytotoxic agents, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

As mentioned above, topical administration may be used. Any common topical formulation such as a solution, suspension, gel, ointment or salve and the like may be employed. Preparation of such topical formulations are described in the art of pharmaceutical formulations as exemplified, for example, by Gennaro et al. (1995) Remington's Pharmaceutical Sciences, Mack Publishing. For topical application, the compositions could also be administered as a powder or spray, particularly in aerosol form. In a some embodiments, the compositions of this invention may be administered by inhalation. For inhalation therapy the active ingredients may be in a solution useful for administration by metered dose inhalers or in a form suitable for a dry powder inhaler. In another embodiment, the compositions are suitable for administration by bronchial lavage.

Suitable formulations for oral administration include hard or soft gelatin capsules, pills, tablets, including coated tablets, elixirs, suspensions, syrups or inhalations and controlled release forms thereof. In another embodiment, the pharmaceutical composition comprises the monoclonal in combination with at least one cytotoxic agent wherein the antibody or agent are in sustained release form. In such formulations, the monoclonal antibody will be distributed throughout the body, prior to, or after release of the cytotoxic agents, allowing for binding of antibody to the cancer cells prior to, or after binding of the cytotoxic agent to the cancer cells. In one embodiment, upon the delayed release of the antibody from such formulations, and subsequent distribution to the site of the cancer cells, the effects of the antibody may be enhanced by the earlier binding or effect of the cytotoxic agent on the cancer cells. Such delayed release formulations may have the same effect as sequential administration of one or more cytotoxic agents followed by the monoclonal antibody or vice versa.

As used herein and unless otherwise indicated, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be, for example, liquids, such as water and oils, including those of petroleum, animal, vegetable or synthetic origin, such as peanut oil, soybean oil, mineral oil, sesame oil and the like. The pharmaceutical vehicles can be saline, methyl cellulose, gum acacia, gelatin, starch paste, talc, keratin, colloidal silica, urea, and the like. In addition, auxiliary, stabilizing, thickening, lubricating and coloring agents may be used. When administered to a patient, the compositions of the invention and pharmaceutically acceptable vehicles are preferably sterile. Water is a preferred vehicle when the composition of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

As used herein and unless otherwise indicated, the phrase “pharmaceutically acceptable salt” includes, but is not limited to, salts of acidic or basic groups that may be present in compositions. Polypeptides included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, (i.e., salts containing pharmacologically acceptable anions), including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate) salts. Polypeptides included in compositions used in the methods of the invention that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein and unless otherwise indicated, the term “pharmaceutically acceptable solvate” means an anti-PSMA monoclonal antibody that further includes a stoichiometric or non-stoichiometric amount of a solvent bound by non-covalent intermolecular forces. Preferred solvents are volatile, non-toxic, and/or acceptable for administration to humans in trace amounts.

As used herein and unless otherwise indicated, the term “pharmaceutically acceptable hydrate” means an anti-PSMA monoclonal antibody that further includes a stoichiometric or non-stoichiometric amount of water bound by non-covalent intermolecular forces.

As used herein and unless otherwise indicated, the term “therapeutically effective” refers to an amount of an anti-PSMA monoclonal antibody, cytotoxic agent or a pharmaceutically acceptable salt, solvate or hydrate thereof able to cause an amelioration of a disease or disorder, or at least one discernible symptom thereof. “Therapeutically effective” also refers to an amount that results in an amelioration of at least one measurable physical parameter, not necessarily discernible by the patient. In yet another embodiment, the term “therapeutically effective” refers to an amount that inhibits the progression of a disease or disorder, either physically (e.g., stabilization of a discernible symptom), physiologically (e.g., stabilization of a physical parameter), or both. In yet another embodiment, the term “therapeutically effective” refers to an amount that results in a delayed onset of a disease or disorder.

As used herein and unless otherwise indicated, the term “prophylactically effective” refers to an amount of an anti-PSMA monoclonal antibody, cytotoxic agent or a pharmaceutically acceptable salt, solvate or hydrate thereof causing a reduction of the risk of acquiring a given disease or disorder. In one embodiment, the compositions are administered as a preventative measure to an animal, preferably a human, having a genetic predisposition to a disorder described herein. In another embodiment of the invention, the compositions are administered as a preventative measure to a patient having a non-genetic predisposition to a disorder disclosed herein. The compositions of the invention may also be used for the prevention of one disease or disorder and concurrently treating another.

The invention also includes isotopically-labeled monoclonal antibodies or binding fragments thereof that have one or more atoms are replaced by an atom having an atomic mass or mass number different from the atomic mass or mass number usually found in nature. Examples of isotopes that can be incorporated into compounds of the invention include isotopes of hydrogen, carbon, fluorine, phosphorous, iodine, copper, rhenium, indium, yttrium, technecium and lutectium (i.e., ³H, ¹⁴C, ¹⁸F, ¹⁹F, ³¹P, ³²P, ³⁵S, ¹³¹I, ¹²⁵I, ¹²³I, ⁶⁴Cu, ¹⁸⁷Re, ¹¹¹In, ⁹⁰Y, ^{99m}Tc, ¹⁷⁷Lu). In some embodiments, isotopes which are metals (e.g., copper, rhenium, indium, yttrium, technecium and lutectium) are non-covalently attached to the monoclonal antibody by chelation. Examples of chelation included in the invention are chelation of a metal isotope to a polyHis region fused to the monoclonal antibody or a binding fragment thereof. Non-metal isotopes may be covalently attached to the monoclonal antibody or a binding fragment thereof using any means acceptable. Other chelation agents include, but are not limited to, DOTA and meDOTA chelates disclosed in U.S. Pat. Nos. 5,435,990 and 5,652,361 both of which are herein incorporated by reference in their entirety.

Antibodies to PSMA, including 7e11, may be conjugated or attached to, or operatively associated with, cytotoxic agents or radioisotopes to prepare immunotoxins. Immunoconjugate technology is now generally known in the art. However, certain advantages may be achieved through the application of certain preferred technology, both in the preparation and purification for subsequent clinical administration. For example, while IgG based constructs will typically exhibit better binding capability and slower blood clearance than their Fab′ counterparts, Fab′ fragment-based constructs will generally exhibit better tissue penetrating capability.

Additionally, while numerous types of disulfide-bond containing linkers are known that can be successfully employed in antibody and peptide conjugation, certain linkers will generally be preferred over other linkers, based on differing pharmacological characteristics and capabilities. For example, linkers that contain a disulfide bond that is sterically hindered are to be included in the invention, due to their greater stability in vivo, thus preventing release of the coagulant prior to binding at the site of action.

Each type of cross-linker, as well as how the cross-linking is performed, will tend to vary the pharmacodynamics of the resultant conjugate. One may desire to have a conjugate that will remain intact under conditions found everywhere in the body except the intended site of action, at which point it is desirable that the conjugate have good release characteristics. Therefore, the particular cross-linking scheme, including in particular the particular cross-linking reagent used and the structures that are cross-linked, will be of some significance.

Depending on the specific agents to be conjugated, it may be necessary or desirable to provide a peptide spacer operatively attaching the antibody and the cytotoxic agent. Certain peptide spacers are capable of folding into a disulfide-bonded loop structure. Proteolytic cleavage within the loop would then yield a heterodimeric polypeptide wherein the antibody and the therapeutic agent are linked by only a single disulfide bond. An example of such a toxin is a Ricin A-chain toxin.

When certain other toxin compounds are utilized, a non-cleavable peptide spacer may be provided to operatively attach the antibody and the toxin compound of the fusion protein. Toxins which may be used in conjunction with non-cleavable peptide spacers are those which may, themselves, be converted by proteolytic cleavage, into a cytotoxic disulfide-bonded form. An example of such a toxin compound is a Pseudonomas exotoxin compound.

A variety of chemotherapeutic and other pharmacological agents have now been successfully conjugated to antibodies and shown to function pharmacologically. Exemplary antineoplastic agents that have been investigated include doxorubicin, daunomycin, methotrexate, vinblastine, and various others. Moreover, the attachment of other agents such as neocarzinostatin, macromycin, trenimon and alpha-amanitin has been described. These attachment methods can be adapted for use herewith.

Any covalent linkage to the antibody should ideally be made at a site distinct from the functional site(s). The compositions are thus linked in any operative manner that allows each region to perform its intended function without significant impairment, in particular, so that the resultant construct still binds to the intended antigen and so that the attached agent substantially maintains biological activity and/or recovers biological activity when released from the construct.

Attachment of biological agents via the carbohydrate moieties on antibodies is also contemplated. Glycosylation, both O-linked and N-linked, naturally occurs in antibodies. Recombinant antibodies can be modified to recreate or create additional glycosylation sites if desired, which is simply achieved by engineering the appropriate amino acid sequences (such as Asn-X-Ser, Asn-X-Thr, Ser, or Thr) into the primary sequence of the antibody.

In additional to the general information provided above, antibodies may be conjugated to therapeutic or other agents using certain preferred biochemical cross-linkers. Cross-linking reagents are used to form molecular bridges that tie together functional groups of two different molecules. To link two different proteins in a step-wise manner, hetero-bifunctional cross-linkers can be used that eliminate unwanted homopolymer formation.

Hetero-bifunctional cross-linkers contain two reactive groups: one generally reacting with primary amine group (e.g., N-hydroxy succinimide) and the other generally reacting with a thiol group (e.g., pyridyl disulfide, maleimides, halogens). Through the primary amine reactive group, the cross-linker may react with the lysine residue(s) of one protein (e.g., the selected antibody or fragment thereof) and through the thiol reactive group, the cross-linker, already tied up to the first protein, reacts with the cysteine residue (free sulfhydryl group) of the other protein.

Compositions therefore generally have, or are derivatized to have, a functional group available for cross-linking purposes. This requirement is not considered to be limiting in that a wide variety of groups can be used in this manner. For example, primary or secondary amine groups, hydrazide or hydrazine groups, carboxyl alcohol, phosphate, carbamate, or alkylating groups may be used for binding or cross-linking.

The spacer arm between the two reactive groups of a cross-linkers may have various length and chemical compositions. A longer spacer arm allows a better flexibility of the conjugate components while some particular components in the bridge (e.g., benzene group) may lend extra stability to the reactive group or an increased resistance of the chemical link to the action of various aspects (e.g., disulfide bond resistant to reducing agents). The use of peptide spacers, such as L-Leu-L-Ala-L-Leu-L-Ala, is also contemplated.

It is preferred that a cross-linker having reasonable stability in serum or blood will be employed. Numerous types of disulfide-bond containing linkers are known that can be successfully employed in conjugation. Linkers that contain a disulfide bond that is sterically hindered may prove to give greater stability in vivo, preventing release of the agent prior to binding at the site of action. These linkers are thus one preferred group of linking agents.

One example of a cross-linking reagents is SMPT, which is a bifunctional cross-linker containing a disulfide bond that is sterically hindered by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the tumor site. It is contemplated that the SMPT agent may also be used in connection with the conjugates of this invention.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers can also be employed in accordance herewith. Other useful cross-linkers, not considered to contain or generate a protected disulfide, include SATA, SPDP and 2-iminothiolane. The use of such cross-linkers is well understood in the art.

Once conjugated, the conjugate is separated from unconjugated antibodies or peptides and other agents and from other contaminants. A large a number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

The invention also includes monoclonal antibodies or binding fragments thereof labeled with a metal such as gadolinium (Gd). In some embodiments, a metal such as gadolinium is covalently attached to the monoclonal antibody by chelation. Examples of chelation included in the invention are chelation of a metal such as gadolinium to a polyHis region fused to a monoclonal antibody.

The methods of the invention also include use of monoclonal antibodies in conjunction with a broad spectrum of cytotoxic agents including cytotoxins. As used herein, “cytotoxins” are any agent which acts on a cell to damage and kill a cell. Examples include, but are not limited to, venoms (e.g., venom phospholipases and microbial toxins); and protein synthesis inhibitors (e.g., diphtheria toxin and toxic plant protein; enzymes that inhibit the action of eukaryotic ribosomes (e.g., ricin, ricin A chain and pokeweed antiviral protein).

The methods used for binding the cytotoxin to the monoclonal antibody molecule can involve either non-covalent or covalent linkages as described herein. Since non-covalent bonds are more likely to be broken before the antibody complex reaches the target site, covalent linkages are preferred. For instance, carbodiimide can be used to link carboxy groups of the pharmaceutical agent to amino groups of the antibody molecule. Bifunctional agents such as dialdehydes or imidoesters can be used to link the amino group of a drug to amino groups of the antibody molecule. The Schiff base reaction can be used to link drugs to antibody molecules. This method involves the periodate oxidation of a drug or cytotoxin that contains a glycol or hydroxy group, thus forming an aldehyde which is then reacted with the antibody molecule. Attachment occurs via formation of a Schiff base with amino groups of the antibody molecule. Additionally, drugs with reactive sulfhydryl groups have been coupled to antibody molecules.

All agents of the present invention, prodrugs thereof, and pharmaceutically acceptable salts of said agents or of said prodrugs which contain the aforementioned isotopes and/or other isotopes of other atoms are within the scope of this invention. In some instances, Indium, Trititium and carbon-14 isotopes are particularly preferred for their ease of preparation and detectability. Further, substitution with heavier isotopes such as deuterium can afford certain therapeutic advantages resulting from greater metabolic stability, for example increased in vivo half-life or reduced dosage requirements and, hence, may be preferred in some circumstances.

### Methods of Treatment Using Cytotoxic Agents

This invention also includes methods for the treatment of cancer in a mammal, including a human, comprising administering to said mammal an amount of a cytotoxic agent, or a pharmaceutical composition comprising an amount of the cytotoxic agent, that is effective in enhancing the binding of a monoclonal antibody to an epitope on the cytoplasmic domain of PSMA when administered prior to or simultaneously with, the monoclonal antibody.

Such methods include the treatment or inhibition of abnormal growth and/or proliferation of cancer cells including malignant cells of neoplastic diseases. Inhibition of abnormal cell growth can occur by a variety of mechanism including, but not limited to, apoptosis, cell death, inhibition of cell division, transcription, translation, transduction, etc. In one embodiment, the cytotoxic agent damages and/or disrupts the cell membrane of the cancer cell, resulting in the exposure of the cytoplasmic domain of PSMA. Simultaneous or subsequent administration of the monoclonal antibody results in binding to the epitope in the cytoplasmic domain and provides a means for eliminating the damaged cancer cells and/or targeting the surrounding cancer cells in a solid tumor.

As discussed above, anti-PSMA monoclonal antibodies or binding fragments thereof can be provided in combination, or in sequential combination with cytotoxic agents that are useful in the treatment of cancer. As used herein, two agents are said to be administered in combination when the two agents are administered simultaneously or are administered independently in a fashion such that the agents will act in an additive or synergistic fashion. For example, monoclonal antibodies can be used in combination with one or more chemotherapeutic agents selected from the following types of chemotherapeutic agents including, but not limited to, apoptotic agents, mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens as described herein. Preferred chemotherapeutic agents induce cellular apoptosis and/or increase binding of an anti-PSMA antibody to a malignant cell as described herein.

In practicing the methods of this invention, an anti-PSMA monoclonal antibody may be used alone or in combination with other therapeutic or diagnostic agents. In certain preferred embodiments, the monoclonal antibody may be co-administered along with other chemotherapeutic agents typically prescribed for various types of cancer according to generally accepted oncology medical practice. The compositions of this invention can be utilized in vivo, ordinarily in mammals, such as humans, sheep, horses, cattle, pigs, dogs, cats, rats and mice or in vitro. The invention is particularly useful in the treatment of human subjects.

### Methods of Treatment Using Radiation

The invention includes a therapeutic method comprising administration of an anti-PSMA monoclonal antibody in combination with radiation for the treatment of cancer. In particular, the radiation is designed to disrupt the cell membrane of the cancer cell to expose the cytoplasmic domain of PSMA as described herein. Once the cytoplasmic domain is exposed, the monoclonal antibody can bind to PSMA and can be used to target the solid tumor with additional radioisotopes which are associated with the antibody. The methods of the invention are also designed to induce apoptosis (cell death) in cancer cells, reduce the incidence or number of metastases, and reduce tumor size. Tumor cell resistance to radiotherapy agents represents a major problem in clinical oncology. Thus, in the context of the present invention, it also is contemplated that combination therapy with such a monoclonal antibody could be used on radiation resistant tumors to improve the efficacy of the radiation therapy.

As discussed above, the invention includes a method of treating cancer comprising administering to a mammal with cancer an amount of an anti-PSMA monoclonal antibody in combination with ionizing radiation, both in sufficient doses that, when combined, cancer cell death is induced. In one embodiment, the presence of the monoclonal antibody reduces the amount of radiation required to treat the cancer when compared to radiation treatment alone. The monoclonal antibody can be provided prior to said radiation, after said radiation or concurrent with said radiation.

Radiation that causes DNA damage has been used extensively and includes what are commonly known as gamma-rays, e-beam, X-rays (e.g., external beam radiation generated by a linear accelerator), and the directed delivery of radioisotopes to tumor cells. It is most likely that all of these factors effect a broad range of damage on DNA, on the precursors of DNA, on the replication and repair of DNA, and on the assembly and maintenance of chromosomes. For external beam radiation treatment in combination with the monoclonal antibody, treatment is usually given as one treatment per day. Occasionally two treatments per day will be given, where a day has been missed, or with certain cancer therapy indications. The standard dosing ranges from about 1.8 Gy to about 2.0 Gy per day, with weekly doses ranging from about 9 Gy to about 10 Gy per week. Treatment is usually given five days per week with two days off for recovery time from the preceding week of treatment.

### Methods of Diagnosis

The invention includes diagnostic methods to detect cancer and/or assess the effect of cytotoxic agents on cancer cells in an organ or body area of a patient. The present methods include administration of a composition comprising a detectable amount of an anti-PSMA monoclonal antibody to a patient before and after treatment with a cytotoxic agent. Following initial administration of the monoclonal antibody the cancer cells can be imaged and the relative amount of cancerous cells determined by any available means. Subsequent to administration of the cytotoxic agent, an additional amount of detectable monoclonal antibody can be administered to determine the relative amount of cancer cells remaining following treatment. Comparison of the before and after treatment images can be used as a means to assess the efficacy of the treatment wherein a decrease in the number of cancer cells imaged following treatment is indicative of an efficacious treatment regimen.

As used herein, the term “detectable amount” refers to the amount of labeled monoclonal antibody which binds to PSMA administered to a patient that is sufficient to enable detection of binding of the labeled monoclonal antibody to one or more malignant cancer cells in a tumor. As used herein, the term “imaging effective amount” refers to the amount of the labeled monoclonal antibody administered to a patient that is sufficient to enable imaging of binding of the monoclonal antibody to one or more malignant cancer cells in a tumor.

The methods of the invention may employ isotopically-labeled monoclonal antibodies which, in conjunction with non-invasive neuroimaging techniques such as magnetic resonance spectroscopy (MRS) or imaging (MRI), or gamma imaging such as positron emission tomography (PET) or single-photon emission computed tomography (SPECT), are used to identify and quantify abnormal cells in vivo including malignant cells in tumors. The term “in vivo imaging” refers to any method which permits the detection of labeled monoclonal antibody as described above. For gamma imaging, the radiation emitted from the tumor or area being examined is measured and expressed either as total binding, or as a ratio in which total binding in one tissue is normalized to (for example, divided by) the total binding in another tissue or the entire body of the same subject during the same in vivo imaging procedure. Total binding in vivo is defined as the entire signal detected in a tumor or tissue by an in vivo imaging technique without the need for correction by a second injection of an identical quantity of labeled compound along with a large excess of unlabeled, but otherwise chemically identical compound. As used herein, the terms “subject” or “patient” refers to a mammal, preferably a human, and most preferably a human suspected of having abnormal cells, including malignant cells in a tumor.

For purposes of in vivo imaging, the type of detection instrument available is a major factor in selecting a given label. For instance, radioactive isotopes are particularly suitable for in vivo imaging in the methods of the present invention. The type of instrument used will guide the selection of the radioisotope. For instance, the radioisotope chosen must have a type of decay detectable by a given type of instrument. Another consideration relates to the half-life of the radioisotope. The half-life should be long enough so that it is still detectable at the time of maximum uptake by the target, but short enough so that the host does not sustain deleterious radiation. The isotopically-labeled monoclonal antibody can be detected using gamma imaging where emitted gamma irradiation of the appropriate wavelength is detected. Methods of gamma imaging include, but are not limited to, positron emission tomography (PET) imaging or for single photon emission computerized tomography (SPECT). Preferably, for SPECT detection, the chosen radiolabel will lack a particulate emission, but will produce a large number of photons. For PET detection, the radiolabel will be a positron-emitting radioisotope which will be detected by the PET camera.

In the present invention, monoclonal antibodies are made which are useful for in vivo detection and imaging of tumors. These compounds are to be used in conjunction with non-invasive neuroimaging techniques such as magnetic resonance spectroscopy (MRS) or imaging (MRI), positron emission tomography (PET), and single-photon emission computed tomography (SPECT). In accordance with this invention, monoclonal antibody may be labeled with any acceptable radioisotope described above by general organic chemistry techniques known to the art (see March (1992) Advanced Organic Chemistry: Reactions, Mechanisms & Structure, Wiley). The monoclonal antibody also may be radiolabeled with isotopes of copper, fluorine, carbon, bromine, etc. for PET by techniques well known in the art and are described (see Phelps (1986) Positron Emission Tomography and Autoradiography, Raven Press pages 391-450). The monoclonal antibody also may be radiolabeled with acceptable isotopes such as iodine for SPECT by any of several techniques known to the art (see Kulkarni (1991) Int. J. Rad. Appl. Inst. 18, 647-648).

For example, the monoclonal antibody may be labeled with any suitable radioactive iodine isotope, such as, but not limited to ¹³¹I by iodination of a diazotized amino derivative directly via diazonium iodide (see Greenbaum (1936) Am. J. Pharm. 108, 17-18), or by conversion of the unstable diazotized amine to the stable triazene, or by conversion of a non-radioactive halogenated precursor to a stable tri-alkyl tin derivative which then can be converted to the iodo compound by several methods well known to the art (see Chumpradit et al. (1991) J. Med. Chem. 34, 877-878 and Zhuang et al. (1994) J. Med. Chem. 37, 1406-1407).

The monoclonal antibody also may be radiolabeled with known metal radiolabels, such as ⁶⁴Cu or ^{99m}Tc. Modification of the substituents to introduce ligands that bind such metal ions can be effected without undue experimentation by one of ordinary skill in the radiolabeling art including covalent attachment to a polyHis region in a modified monoclonal antibody. The metal radiolabeled monoclonal antibody can then be used to detect and image tumors.

The diagnostic methods of the present invention may use isotopes detectable by nuclear magnetic resonance spectroscopy for purposes of in vivo imaging and spectroscopy. Elements particularly useful in magnetic resonance spectroscopy include, but are not limited to, ¹⁹F and ¹³C. Suitable radioisotopes for purposes of this invention include beta-emitters, gamma-emitters, positron-emitters and x-ray emitters. These radioisotopes include, but are not limited to, ¹⁷⁷Lu, ¹¹¹In, ¹³¹I, ¹²³I, ¹⁸F, ¹¹C, ⁷⁵Br and ⁷⁶Br.

Suitable stable isotopes for use in Magnetic Resonance Imaging (MRI) or Spectroscopy (MRS), according to this invention include, but are not limited to, ¹⁹F and ¹³C. Suitable radioisotopes for in vitro identification and quantification of abnormal cells including tumor cells, in a tissue biopsy or post-mortem tissue include ¹²⁵I, ¹⁴C and ³H. The preferred radiolabels are ⁶⁴Cu or ¹⁸F for use in PET in vivo imaging, ¹²³I or ¹³¹I for use in SPECT imaging in vivo, ¹⁹F for MRS and MRI and ³H or ¹⁴C for in vitro methods. However, any conventional method for visualizing diagnostic probes can be utilized in accordance with this invention.

Generally, the dosage of the isotopically-labeled monoclonal antibody will vary depending on considerations such as age, condition, sex, and extent of disease in the patient, contraindications, if any, concomitant therapies and other variables, to be adjusted by the skilled artisan. Dosage can vary from 0.001 mg/kg to 1000 mg/kg, preferably 0.1 mg/kg to 100 mg/kg. Administration to the patient may be local or systemic and accomplished intravenous, intra-arterial, intra-thecal (via the spinal fluid), intra-cranial or the like. Administration may also be intra-dermal or intra-cavitary, depending upon the body site under examination.

After a sufficient time has elapsed for the labeled monoclonal antibody to bind with the abnormal cells, for example thirty minutes to forty-eight hours, the area of the subject under investigation is examined by routine imaging techniques such as MRS/MRI, SPECT, planar scintillation imaging, PET, and emerging imaging techniques, as well. The exact protocol will necessarily vary depending upon factors specific to the patient, as noted above, and depending upon the body site under examination, method of administration and type of label used; the determination of specific procedures would be routine to the skilled artisan. For tumor imaging, preferably, the amount (total or specific binding) of the bound isotopically-labeled monoclonal antibody is measured and compared (as a ratio) with the amount of isotopically-labeled monoclonal antibody bound to the tumor following chemotherapeutic treatment.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Anti-PSMA-meO-DOTA Immunoconjugate (CYT-500)

Anti-PSMA-meO-DOTA Immunoconjugate (CYT-500) is comprised of 7E11C5-3 monoclonal antibody (CYT-351) that is currently used in the manufacture of its commercial product ProstaScint®. ProstaScint® is comprised of CYT-351 conjugated via periodate oxidation of the carbohydrate groups located on the heavy chains to the linker-chelator GYK-DTPA HCl [glycyl-tyrosyl-(N-ε-diethylenetriaminepentaacetic acid)-lysine hydrochloride] which is complexed with the gamma emitting radioisotope ¹¹¹In. Anti-PSMA-meO-DOTA Immunoconjugate is comprised of CYT-351 covalently conjugated to the linker-chelator meO-DOTA [α-(5-isothiocyanato-2-methoxyphenyl)-1,4,7,10-tetraazacyclododecane-1,4,7,10-tetraacetic acid].

The CYT-351-meO-DOTA immunoconjugate has been shown to be stable in human serum thereby decreasing the chance for secondary toxicities as a result of shed linker and/or radioisotopes.

### Example 2

### 7E11C5-3 Monoclonal Antibody (CYT-351)

CYT-351 is a murine IgG1 monoclonal antibody secreted by a murine/murine hybridoma cell line, which was produced by immunizing BALB/c mice with live LNCaP human prostatic adenocarcinoma cells and partially purified LNCaP plasma membranes. The LNCaP cell line used to immunize the mice is a well characterized continuous cell line which was established from a needle biopsy taken from a lymph node metastasis of human prostatic adenocarcinoma. LNCaP cells grow readily in vitro, form clones in semisolid media, show an aneuploid (modal number 76-91) human male karyotype with several marker chromosomes and maintain the malignant properties of an adenocarcinoma.

The CYT-351 hybridoma was established and originally described by Horoszewicez et al. (1987) Anticancer Res. 7, 927-936 and U.S. Pat. Nos. 5,162,504 and 5,578,484). Spleen cells from mice immunized with live LNCaP cells were fused with P3X63Ag8.653 murine myeloma cells. The cells were cloned twice by limited dilution cloning and a stable hybridoma, designated hybridoma 7E11-C5, was expanded and cryopreserved. This clone secreted a prostate-specific monoclonal antibody of the IgG1 subclass which was originally designated monoclonal antibody 7E11-C5.

A culture of the CYT-351 seed stock was used to establish a 100 vial Master Cell Bank (MCB). A single vial of cells was thawed and the cells recovered into a 25 cm² flask containing basal cell culture medium supplemented with 2.5% FBS (fetal bovine serum). The cells were subsequently expanded into 75 cm² flasks, 150 cm² flasks, a 500 ml spinner flask, and finally on into a three liter spinner. The cells were harvested and placed into freezing medium (basal medium supplemented with 20% FBS and 10% DMSO). The cells were then aliquoted into 100 vials, each containing approximately 9×10⁶ cells and labeled with the designation 2MM0180-M001-9M, and subsequently stored in the vapor phase of liquid nitrogen. Ten vials from the serum-grown MCB were used for tests to determine if the preparation was sterile and free of infectious adventitious agents. The results of these tests demonstrated that the CYT-351 MCB was sterile and free of infectious adventitious agents

### Example 3

### Methoxy-DOTA Linker

Methoxy-DOTA (α-(5-isothiocyanato-2-methoxyphenyl)-1,4,7,10-tetraazacyclododecane-1,4,7,10-tetraacetic acid) is prepared from a purely synthetic process. meDOTA and its methods of use and manufacture is disclosed in U.S. Pat. Nos. 5,435,990 and 5,652,361 both of which are herein incorporated by reference in their entirety.

### Example 4

### CYT-351 Manufacturing Process

The cell banks, components, raw materials and manufacturing process used to produce CYT-351 intermediate antibody for use in producing Anti-PSMA-meO-DOTA Immunoconjugate (CYT-500) are done so in accordance with the GMP manufacturing process.

The growth/production medium for the CYT-351 hybridoma is a defined, serum-free media available from HyClone Laboratory (HyQ-CCM™) and is comprised of 925 basal medium. Cell culture is performed in an AcuSyst-Xcell hollow fiber bioreactor and pH, temperature and oxygen levels monitored throughout the run. Samples are removed to monitor glucose, lactate and CYT-351 levels. Media feed is achieved via peristaltic pump. Medium is perfused through the bioreactor and the conditioned medium containing CYT-351 is harvested, clarified by filtration and stored at 2 to 8° C. The production run typically lasts for 60 to 70 days.

Each CYT-351 harvest is sampled and tested for CYT-351 titer, immunoreactivity, endotoxin and bioburden. Prior to purification, pooled harvest samples are tested minimally for: CYT-351 concentration, Mycoplasma, sterility and virus by reverse transcriptase, XC Plaque, S+L-Focus and in vitro viral testing.

Harvest and purification of CYT-351 are performed in classified rooms with appropriate environmental monitoring to allow for aseptic processing. The CYT-351 harvest is filtered through a 0.45 μm filter, concentrated to approximately 6 to 12 mg/ml CYT-351 using a Pellicon tangential-flow ultrafiltration device fitted with a 30 kDa cutoff membrane. Following concentration, the concentrated crude CYT-351 product is passed over a Sephadex G-25 column to remove low molecular weight moieties. The G-25 column is equilibrated and eluted with 0.7 M ammonium sulfate (pH 8.0 to 8.4).

The eluted protein (CYT-351) peak is loaded onto a Protein A affinity column equilibrated with 0.7 M ammonium sulfate. The loaded Protein A column is washed with thirty (30) column volumes of 0.7 M ammonium sulfate followed by a short wash with 55 mM sodium acetate (pH 7.0 to 8.5). Bound CYT-351 is eluted from the Protein A column with 55 mM sodium acetate (pH 4.0 to 4.5) and the pH of the eluted product adjusted to 5.1 to 5.3 with 55 mM sodium acetate (pH 7.0 to 8.5).

The Protein A purified material is passed over a DEAE Sepharose column equilibrated in 55 mM sodium acetate (pH 5.1 to 5.3). This is a passive purification step in that the CYT-351 passes over the column whereas DNA, albumin and other acidic components bind to the support.

The CYT-351 peak is then loaded onto a S-Sepharose column equilibrated with 55 mM sodium acetate (pH 5.1 to 5.3). The column is washed with 10 mM sodium phosphate buffer (pH 5.9 to 6.1). The bound CYT-351 is eluted with 10 mM phosphate buffered saline (pH 5.9 to 6.1). Purified CYT-351 is filtered through a sterile 0.22 μm, sampled for Quality Control testing and stored at 2 to 8° C. until needed for conjugation. Sterile filtered bulk CYT-351 has an approved shelf life of three years at 2 to 8° C.

### Example 5

### Manufacturing Process for the Immunoconjugate CYT-500

Prior to conjugation, the purified CYT-351 is passed through a DV-20 (PALL) virus removal filter. The commercial manufacturing process for CYT-351, described above, results in 8.9 log viral removal. An additional 5 to 6 log viral removal is obtained using the DV-20 filter, resulting in approximately 14 log removal.

Purified monoclonal antibody CYT-351 is combined with 0.22 μm filtered (cellulose acetate) meO-DOTA in 0.5 M HEPES (pH 8.85). The linker to CYT-351 ratio is 70:1 with a total of 6 grams CYT-351 used for the toxicology lot (clinical lots also are 6 gram CYT-351 scale). The reaction mixture is incubated for three hours at 35 to 37° C. with gentle stirring. Following three hours, the reaction mixture is adjusted to 7.0 with 1 M acetic acid to slow the reaction. The resultant product was concentrated from approximately 1900 to 300 ml using a Millipore Labscale TFF system with one Pelicon XL Biomax 50 filter. The concentrate was stored overnight at 2-8° C. The concentrate was chromatographed with 0.1M sodium acetate (pH 5.5) on a 9×90 cm Superose 12 column. The main (product) fraction was collected and concentrated to approximately 21 mg/ml using a Millipore Labscale TFF system with one Pelicon XL Biomax 50 filter. This material (CYT-500) was filtered through a 0.22 μm filter and stored at 2 to 8° C.

The bulk CYT-500 is stored at 2 to 8° C. and tested for contaminants before being released for use. Released CYT-351 is filtered through a sterile 0.22 μm filter and filled into 10 ml Type 1 borosilicate glass vials and stoppered with presterilized 20 mm stoppers. The filled, unlabeled vials are sealed with 20 mm flip-off crimp, visually inspected and sampled for Quality Control testing. Vials are placed in trays marked “quarantine” pending release.

### Example 6

### 7E11-meO-DOTA Serum Stability

An important requirement of antibody-chelating agent immunoconjugates is that they form kinetically inert complexes with metals of interest, in this case, ¹⁷⁷Lu. These complexes must be stable following conjugating to a protein and should stay intact in vivo to avoid secondary toxicities. Similarly, loss of lanthanide metals can result in toxic effects, such as radioactive doses to the liver and bone. Accordingly, we tested serum stability of ¹⁷⁷Lu labeled CYT-500 and compared it to ¹¹¹In-labeled ProstaScint.

Size exclusion chromatography was used to analyze the radioactivity (¹⁷⁷Lu) loss from the complex-conjugate in serum. Uncomplexed ¹⁷⁷Lu associates with serum proteins and tends to elute with the high molecular weight species, similarly to ¹⁷⁷Lu-meO-DOTA-immunoconjugate (¹⁷⁷Lu-CYT-500). The fact that serum proteins bind Lu weakly in a non-specific manner allows us to differentiate between the serum protein ¹⁷⁷Lu complex and ¹⁷⁷Lu-CYT-500. The weak association between ¹⁷⁷Lu and serum proteins can be broken up by DTPA, while DTPA can not transchelate the metal from DOTA type chelates.

To determine if one percent metal loss from the complex conjugate can be measured, mixtures of ¹⁷⁷Lu-CYT-500 and ¹⁷⁷Lu-MeO-DOTA were prepared. The radioactivity in both ¹⁷⁷Lu-CYT-500 and ¹⁷⁷Lu-MeO-DOTA was determined by radioactive counting before mixing them. Two samples were prepared. In the first sample 7% of the total radioactivity came from ¹⁷⁷Lu-MeO-DOTA and in the second one 1%. The size exclusion chromatography analysis showed 9.8 and 3.0% of the radioactivity eluting as the low molecular weight component. The chromatographic method and counting gave the same results within the experimental error.

¹⁷⁷Lu-CYT-500 antibody conjugate was incubated in human serum and before HPLC analysis DTPA was added to the sample to complex nonspecifically bound Lu. The results are tabulated in Table 1. During the two week course of the study insignificant metal loss was observed for ¹⁷⁷Lu-CYT-500 (98% at day 0 and 96% at day 15) and minimal metal loss was observed for ¹¹¹In-DTPA-Cyt-351 (98% at day 1 and 91% at day 15). Radioactivity associated with the high molecular weight components of the mixture, determined by size exclusion chromatography after addition of DTPA.

TABLE 1

| | % Radioactivity associated with high mw | | | | |
|---|---|---|---|---|---|
| Time (day) | ¹⁷⁷Lu | ¹⁷⁷Lu-CYT-500 | SD | ¹¹¹In-ProstaScint | SD |
| 0 | 0 | 97.8 | 0.8 | 98.4 | 0.1 |
| 1 | 0 | 98.8 | 0.1 | 97.1 | 0.1 |
| 2 | 0 | 98.5 | 0.4 | 97.2 | 0.1 |
| 3 | 0 | 96.5 | 0.2 | 97.1 | 0.1 |
| 4 | 0 | 97.7 | 0.2 | 96.2 | 0.6 |
| 6 | 0 | 97.1 | 0.7 | 96.1 | 0.7 |
| 7 | 0 | 97.9 | 0.5 | 95.1 | 1.1 |
| 8 | 0 | 98.2 | 0.1 | 94.7 | 0.6 |
| 9 | 0 | 97.0 | 0.5 | 94.6 | 1.1 |
| 10 | 0 | 98.1 | | 94.2 | 1.1 |
| 15 | 0 | 95.6 | 0.4 | 91.0 | 0.4 |

### Example 7

### 7E11-meO-DOTA Acute Toxicity Study in Rats

The purpose of this study was to determine the potential toxicity (including neurotoxicity) of Anti-PSMA-meO-DOTA Immunoconjugate (CYT-500) when administered once by intravenous injection to male Sprague Dawley rats. Eighty male rats were randomly assigned to one of four groups and administered 100 mM sodium acetate buffer (control article) or Anti-PSMA-meO-DOTA Immunoconjugate (CYT-500) at 3, 15 or 30 mg/kg once on Study Day (SD). Forty rats (10/group) were subjected to a full gross necropsy on SD 4; the remaining rats were necropsied on SD 15. An additional 27 rats were assigned to one of the three treated groups (9/group) and blood was collected at selected timepoints for future toxicokinetic profiling.

Parameters evaluated included mortality, clinical observations, body weight, food consumption, neurotoxicity, ophthalmology, clinical pathology, gross pathology, absolute and relative organ weights and histopathology. Treatment with Anti-PSMA-meO-DOTA Immunoconjugate (CYT-500) had no effect on mortality, clinical observations, body weight, food consumption, neurotoxicity, ophthalmology, clinical pathology, gross pathology, absolute and relative organ weights and histopathology. Therefore, under the conditions of this study the observed no-effect level (NOEL) is at least 30 mg/kg (100× the anticipated human dose).

### Example 8

### 7E11-meO-DOTA Acute Toxicity Study in Dogs

The purpose of this study was to determine the potential toxicity of Anti-PSMA-meO-DOTA Immunoconjugate (CYT-500) when administered once by intravenous injection to male beagle dogs. Twenty four male dogs were randomly assigned to one of four groups and administered 100 mM sodium acetate buffer (control article) or Anti-PSMA-meO-DOTA Immunoconjugate (CYT-500) at 0.6, 3 or 6 mg/kg once on SD 1. Twelve dogs (three per group) were subjected to a full gross necropsy on SD 4; the remaining 12 dogs were necropsied on SD 15. Parameters evaluated included mortality, clinical observations, body weights, food consumption, ophthalmology, cardiology, clinical pathology, gross pathology, absolute and relative organ weights, and histopathology.

Treatment with Anti-PSMA-meO-DOTA Immunoconjugate (CYT-500) had no effect on mortality, clinical observations, body weights, food consumption, ophthalmology, cardiology, clinical pathology, gross pathology or absolute and relative organ weights. Test article related findings consisted of vasculitis of the central veins of the liver in treated animals. Lesions were more pronounced in SD 4 animals and, although present, appeared to be resolving in SD 15 animals. The most severe lesions in SD 4 animals were seen in animals treated at 3 or 6 mg/kg (10 and 20× the anticipated human dose, respectively). By SD 15, the lesions were milder overall, suggesting that with additional time resolution may be possible. In conclusion, intravenous injections of Anti-PSMA-meO-DOTA Immunoconjugate (CYT-500) were generally well tolerated.

### Example 9

### 7E11-meO-DOTA Cardiovascular Safety Pharmacology Study

The purpose of this study was to evaluate cardiovascular safety following intravenous administration of Anti-PSMA-meO-DOTA Immunoconjugate (CYT-500) in male Beagle dogs. Seven male dogs were given an intravenous injection of 100 mM sodium acetate buffer on SD 1, and Anti-PSMA-meO-DOTA Immunoconjugate (CYT-500) at 0.6 mg/kg on SD 8, 3 mg/kg on SD 15, and 6 mg/kg on SD 22 and 29. Each dose administration was followed by at least a one-week wash-out period. Cardiovascular profiling and body temperature data were collected via telemetry following doses on SD 1, 8, 15 and 22. Other parameters evaluated included mortality, clinical observations, and body weights.

Treatment with Anti-PSMA-meO-DOTA Immunoconjugate (CYT-500) at doses up to 6 mg/kg had no effects on blood pressure, heart rate, electrocardiographic parameters, body temperature, body weights or mortality. One animal experienced anaphylaxis shortly after administration of a 6 mg/kg dose on SD 22. This animal was removed from the study and returned to the stock colony. Symptoms of anaphylaxis were not observed in any other animals following both a single and repeat dose at 6 mg/kg. In conclusion, intravenous injection of Anti-PSMA-meO-DOTA Immunoconjugate (CYT-500) at doses up to 6 mg/kg were generally well tolerated.

### Example 10

### 7E11-meO-DOTA Respiratory Function Study

The purpose of this study was to evaluate respiratory function following intravenous administration of Anti-PSMA-meO-DOTA Immunoconjugate (CYT-500) in male Beagle dogs. Six male dogs were given an intravenous injection of 100 mM sodium acetate buffer on SD 1, and Anti-PSMA-meO-DOTA Immunoconjugate (CYT-500) at 6 mg/kg on SD 4. Parameters evaluated included mortality, clinical observations, body weights and respiratory function assessment. Respiratory function assessment included respiratory rate, saturated blood oxygen levels (SpO₂) and end-tidal pressures (ETCO₂).

Treatment with Anti-PSMA-meO-DOTA Immunoconjugate (CYT-500) had no effect on mortality, clinical observations, body weight, or respiratory function. Therefore under the conditions of this study the no-observed effect-level (NOEL) is at least 6 mg/kg.

Although the present invention has been described in detail, it is understood that various modifications can be made without departing from the spirit of the invention. Accordingly, the invention is limited only by the following claims. All cited patents, patent applications and publications referred to in this application are herein incorporated by reference in their entirety.

1. A method for treating cancer which comprises a malignant cell expressing PSMA in a patient in need thereof comprising administering a monoclonal antibody or antigen binding fragment thereof which specifically binds to a cytoplasmic epitope on PSMA in combination with at least one cytotoxic agent.

2. The method of claim 1 wherein the cytotoxic agent is administered prior to administration of the monoclonal antibody.

3. The method of claim 1 wherein the cytotoxic agent is administered simultaneously with the monoclonal antibody.

4. The method of claim 1 wherein the antibody is linked to a cytotoxic agent.

5. A method of imaging a tumor in a patient comprising administering a cytotoxic agent followed by administration of a monoclonal antibody which specifically binds to a cytoplasmic epitope on PSMA expressed by a malignant cell.

6. The method of claim 1 wherein the cytotoxic agent disrupts the malignant cell membrane.

7. The method of claim 1 wherein the cytotoxic agent induces cellular apoptosis.

8. The method of claim 1 wherein the monoclonal antibody binds to PSMA expressed by apoptotic endothelial cells.

9. The method of claim 1 wherein the cytotoxic agent is selected from the group consisting of cytotoxins, chemotherapeutic agents and radiation.

10. The method of claim 9 wherein the cytotoxin is selected from the group consisting of gelonin, ricin, saponin, pseudomonas exotoxin, pokeweed antiviral protein, diphtheria toxin and complement proteins.

11. The method of claim 9 wherein the chemotherapeutic agent is selected from the group consisting of alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens.

12. The method of claim 9 wherein the chemotherapeutic agent is selected from the group consisting of BCNU, cisplatin, gemcitabine, hydroxyurea, paclitaxel, temozomide, topotecan, fluorouracil, vincristine, vinblastine, procarbazine, dacarbazine, altretamine, cisplatin, methotrexate, mercaptopurine, thioguanine, fludarabine phosphate, cladribine, pentostatin, fluorouracil, cytarabine, azacitidine, vinblastine, vincristine, etoposide, teniposide, irinotecan, docetaxel, doxorubicin, daunorubicin, dactinomycin, idarubicin, plicamycin, adriamycin, mitomycin, bleomycin, tamoxifen, flutamide, leuprolide, goserelin, aminoglutethimide, anastrozole, amsacrine, asparaginase, mitoxantrone, mitotane and amifostine.

13. The method of claim 9 wherein the radiation is a radioisotope.

14. The method of claim 13 wherein the radioisotope is selected from the group consisting of ³H, ¹⁴C, ¹⁸F, ¹⁹F, ³¹P, ³²P, ³⁵S, 131I, ¹²⁵I, ¹²³I, ⁶⁴Cu, ¹⁸⁷Re, ¹¹¹In, ⁹⁰Y, ^{99m}Tc, ¹⁷⁷Lu.

15. The method of claim 9 wherein the radiation is external beam radiation.

16. The method of claim 1 wherein the cancer comprises a solid tumor.

17. The method of claim 16 wherein the solid tumor is an endothelial cell carcinoma.

18. The method of claim 17 wherein the endothelial cell carcinoma is selected from the group consisting of renal cell carcinoma, colon carcinoma, transitional cell carcinoma, lung carcinoma, breast carcinoma and prostatic adenocarcinoma.

19. The method of claim 18 wherein the renal carcinoma is selected from the group consisting of clear cell carcinoma, papillary carcinoma, chromophobe carcinoma, collecting duct carcinoma and unclassified carcinoma.

20. The method of claim 18 wherein the lung carcinoma is selected from the group consisting of adenocarcinoma, alveolar cell carcinoma, squamous cell carcinoma, large cell and small cell carcinoma.

21. The method of claim 18 wherein the breast carcinoma is selected from the group consisting of adenocarcinoma, ductal carcinoma in situ, lobular carcinoma in situ, invasive ductal carcinoma, medullary carcinoma and mucinous carcinoma.

22. The method of claim 16 wherein the solid tumor is an endothelial cell sarcoma.

23. The method of claim 22 wherein the endothelial cell sarcoma is a soft tissue sarcoma.

24. The method of claim 16 wherein the solid tumor is metastatic.

25. The method of claim 1 wherein the monoclonal antibody is labeled.

26. The method of claim 25 wherein the label is a radiolabel.

27. The method of claim 26 wherein the radiolabel is selected from the group consisting of ³H ¹⁴C, ¹⁸F, ¹⁹F, ³¹P, ³²P, ³⁵S, ¹³¹I, ¹²⁵I, ₁₂₃I, ⁶⁴Cu, ¹⁸⁷Re, ¹¹¹In, ⁹⁰Y, ^{99m}Tc and ¹⁷⁷Lu.

28. The method of claim 27 wherein the radiolabel is ¹⁷⁷Lu.

29. The method of claim 28 wherein the ¹⁷⁷Lu is linked to the antibody by α-(5-isothiocyanato-2-methoxyphenyl)-1,4,7,10-tetraazacyclododecane-1,4,7,10-tetraacetic acid (methoxy-DOTA).

30. The method of claim 29 wherein the antibody is 7e11-C53.

31. The method of claim 1 wherein the monoclonal antibody is 7E11-C53.

32. The method of claim 1 wherein the patient is human.
